(19) United States
(12) Patent Application Publication
(10) Pub. No.: US 2024/0001076 A1
(43) Pub. Date: Jan. 4, 2024

(54) RAPID EXCHANGE EYELET FOR CATHETER

(71) Applicant: KONINKLIJKE PHILIPS N.V., EINDHOVEN (NL)

(72) Inventor: Jacob James GELSINGER, COLORADO SPRINGS, CO (US)

(21) Appl. No.: 18/210,124

(22) Filed: Jun. 15, 2023

Related U.S. Application Data
(60) Provisional application No. 63/357,046, filed on Jun. 30, 2022.

(51) Int. Cl. A61M 25/00; A61M 25/09

(52) U.S. Cl. CPC A61M 25/0043; A61M 25/09041; A61M 2025/006; A61M 2025/0183

(57) ABSTRACT

An intravascular therapy device (10) includes a treatment catheter (12) having a guidewire lumen (14) extending between a guidewire port (16) of the treatment catheter and a distal end (18) of the treatment catheter, a length of the guidewire lumen being less than a length of the treatment catheter; and a plurality of eyelets (24) disposed on an exterior of the treatment catheter and configured to secure an associated guidewire (20) to the treatment catheter.

### CROSS REFERENCE TO RELATED APPLICATIONS

This application claims the benefit of U.S. Provisional Patent Application No. 63/357,046 filed Jun. 30, 2022. This application is hereby incorporated by reference herein.

### FIELD

The following relates generally to the catheter arts, vascular therapy, lesion treatment arts, and related arts.

### BACKGROUND

In catheter-based vascular therapy, a laser catheter utilizes 308 nm laser light transmitted down a catheter to the distal tip where the light can ablate tissue. The laser light can be used with a contrast mixture. The laser light breaks down the contrast and, in the process, generates a sonic wave. This sonic wave emanates in a circular wave around the tip of the laser catheter. When emitted in a calcified lesion, the sonic wave can create fractures in the calcium. However, because of the circular nature of the wave, if there is a specific section that does not have a calcified lesion that fully encapsulates the vessel, then a large portion of the generated energy from the pressure wave may bypass the targeted calcium.

When using a current catheter device, a guidewire exits a rapid exchange port within the patient body and the remaining length of the guidewire runs in tandem with the working length of the catheter. If the guide wire is manipulated separately from the catheter in an incorrect manner the guidewire can wrap itself around the catheter or loop itself into a knot. These events could lead to extreme difficulty of advancing the catheter or removal of the catheter from the body, which could result for the need for surgery.

The following discloses certain improvements to overcome these problems and others.

### SUMMARY

In some embodiments disclosed herein, an intravascular therapy device includes a treatment catheter having a guidewire lumen extending between a guidewire port of the treatment catheter and a distal end of the treatment catheter, a length of the guidewire lumen being less than a length of the treatment catheter; and a plurality of eyelets disposed on an exterior of the treatment catheter and configured to secure an associated guidewire to the treatment catheter.

In some embodiments disclosed herein, an intravascular therapy device includes a treatment catheter having a guidewire lumen extending between a guidewire port of the treatment catheter and a distal end of the treatment catheter, the guidewire lumen providing a path between the guidewire port and the distal end of the treatment catheter, a length of the guidewire lumen being less than one-half of a length of the treatment catheter; and a plurality of eyelets disposed on an exterior of the treatment catheter along a portion of the length of the treatment catheter located between a proximal end of the treatment catheter and the guidewire port.

In some embodiments disclosed herein, an intravascular therapy method includes inserting a guide sheath in a blood vessel of a patient and to an occlusion in the blood vessel; inserting a guidewire through the inserted guide sheath at least up to the occlusion in the blood vessel; inserting a proximal end of the guide wire into a distal end of a treatment catheter, through a guidewire lumen of the treatment catheter, and out a guidewire port of the treatment catheter, wherein a length of the guidewire lumen is less than a length of the treatment catheter; after exiting the guidewire port, threading the proximal end of the guidewire through a plurality of eyelets disposed on an exterior of the treatment catheter; after the threading, sliding the treatment catheter through the guide sheath along the guidewire toward the occlusion in the blood vessel; and after the sliding, performing a therapy on the occlusion using the treatment catheter.

One advantage resides in providing an eyelet to pin a guidewire against a catheter to reduce occurrences of the guidewire wrapping around the catheter.

Another advantage resides in providing an eyelet to pin a guidewire against a catheter to reduce occurrences of the guidewire forming a knot.

Another advantage resides in providing an eyelet to pin a guidewire against a catheter without adding excessive surface contact therebetween.

Another advantage resides in providing an eyelet to allow a physician to use shorter guidewires.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy device in accordance with the present disclosure.

FIG. 1 diagrammatically illustrates another embodiment of the vascular therapy device of FIG. 1.

FIG. 3 diagrammatically illustrates a vascular therapy method using the device of FIG. 1.

### DETAILED DESCRIPTION

In existing intravascular procedures, a crossing guide wire is placed within the vasculature and is navigated to and across the treatment site, then a guide catheter (or guide sheath) is inserted into the incision over the crossing guide wire providing the entry point into the vasculature for other crossing devices. When entering in the leg (contralateral or antegrade approach), the guide catheter is typically 75 cm in length and hence does not reach all the way to the treatment site. For radial entry in the arm, a longer guide catheter of around 120 cm is typically used. Next, a guide wire exchange is potentially performed to insert the specific guide wire size that is compatible with the chosen therapy device. The treatment guidewire is inserted via the guide catheter and is passed completely through the guide catheter and a further distance through the vasculature (typically around 30-70 cm further) to reach and go slightly past the treatment site. Finally, the treatment catheter (e.g., a laser catheter, balloon angioplasty catheter, a catheter bearing a debulking tool, or so forth) is inserted and travels along the guide wire through the guide catheter and to the treatment site, where it is used to perform the treatment.

There are currently two designs for moving the treatment catheter along the guidewire. In the Over the Wire (OTW) approach, the treatment catheter has a guidewire lumen that runs the entire working length of the treatment catheter. This typically requires the guidewire be at least 2×the length of the OTW treatment catheter. By contrast, in the Rapid Exchange (RX) approach, the guidewire lumen runs extends over only the distalmost 10 cm or so of the treatment catheter, and then exits an RX port of the treatment catheter. Hence, when inserting an RX catheter, the guidewire passes through the short (e.g. 10 cm) guidewire lumen then exits the RX port and runs parallel with the treatment catheter for the remainder of its length. The RX approach has certain advantages, including reduced friction between the treatment catheter and the guidewire since the latter only runs through a guidewire lumen of around 10 cm or so, and (as the name suggests) the ability to rapidly exchange treatment catheters. However, a disadvantage of the RX approach is that since the guidewire runs externally to the treatment catheter along most of the length of the treatment catheter, there is potential for the guidewire to loop around the treatment catheter or otherwise get entangled. This is of particular concern for the portion of the treatment catheter extending the final 30 70 cm or so outside the guide catheter.

One way to address this problem would be to extend the length of the guidewire lumen in the RX design; that is, to move the RX port further back from the distal end of the treatment catheter. However, this would increase friction between the guidewire and the treatment catheter, and would also partially mitigate the benefits of the RX design since for example changing out treatment catheters would be more unwieldy.

To address this problem, the following discloses adding eyelets to the outside of the treatment catheter. As the surgeon inserts the treatment catheter, he or she initially feeds the proximal end of the guidewire into the guidewire lumen of the treatment catheter until the proximal end passes out of the RX port. Then, as the treatment catheter continues to be pushed in, the surgeon feeds the guidewire through the eyelets, one after another. This provides for the guidewire to be held close to the outer surface of the treatment catheter, thus reducing likelihood of guidewire looping or entanglement. In an embodiment, the eyelets extend along the distalmost 30 70 cm or so of the treatment catheter that (during treatment) will extend outside of the guide catheter. In some examples, an eyelet spacing of around 5-10 cm between neighboring eyelets would be suitable.

An advantage of this approach is that the friction between the treatment catheter and guidewire is reduced as compared with lengthening the guidewire lumen of the RX catheter. To further reduce the friction, a reduced number of eyelets that are actually used can be chosen based on the expected insertion distance, e.g. the last few eyelets furthest away from the tip of the treatment catheter can remain unused. The physician is able to choose how many eyelets are used based on anatomy and length of guide sheath they are using.

In one approach, the catheter outer surface is PEBAX, and the eyelets are rings of PEBAX. To form the openings of the eyelets a mandrel is passed through them and the PEBAX eyelets are thermally fused to the PEBAX outer surface of the catheter. Alternatively, the loops can be formed as partial cutouts of catheter material itself that are bent to form loops with a mandrel placed through them, and the distal ends of the cutouts are then thermally fused to the catheter. In other embodiments, the eyelets can be metal rings that are secured to the catheter by successive material coatings or the like.

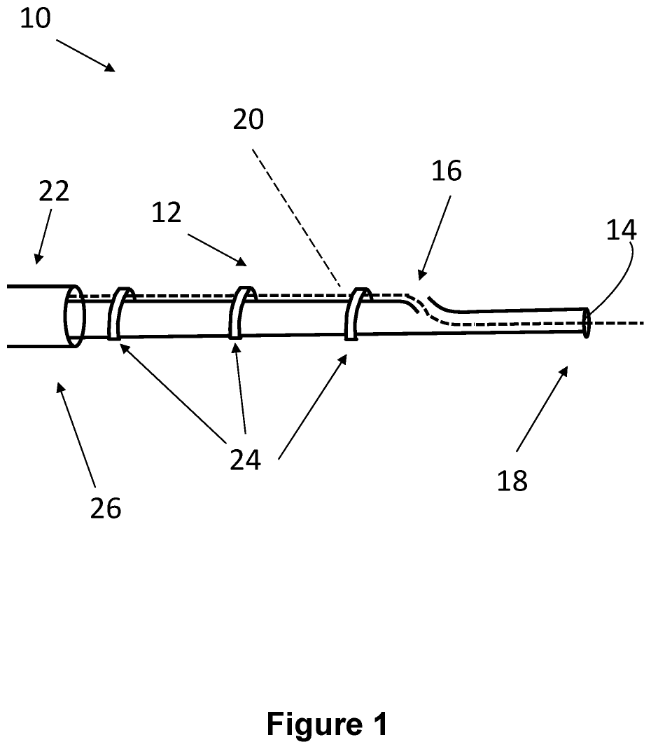

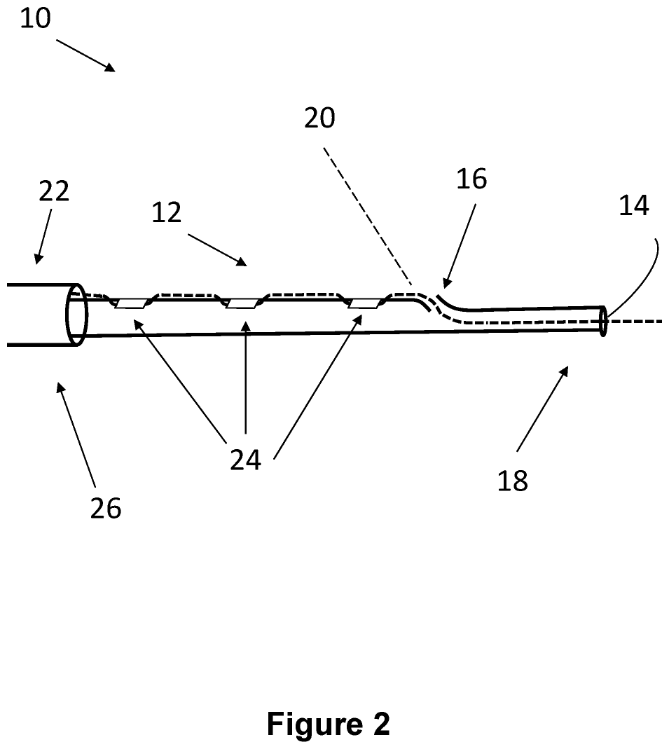

With reference to FIG. 1, an illustrative intravascular therapy device 10 insertable into a blood vessel for treating a lesion (or a clot, or an occlusion, and so forth) in the blood vessel is diagrammatically shown. As shown in FIG. 1, the device 10 includes a treatment catheter 12 including a guidewire lumen 14 extending between a guidewire port 16 and a distal end 18 of the treatment catheter 12. The guidewire port 16 is spaced from the distal end 18 of the treatment catheter 12. The treatment catheter 12 can comprise a plastic, such as PEBAX. The guidewire lumen 14 provides a path between the guidewire port 16 and the distal end 18 of the treatment catheter 12. A length of the guidewire lumen 14 is less than a length of the treatment catheter 12. In some examples, a ratio of the length of the guidewire lumen 14 to the length of the treatment catheter 12 is 0.5 or less. In a particular example, the length of the treatment catheter 12 is at least 30 cm, and the length of the guidewire lumen 14 is 15 cm or less.

The treatment catheter 12 is configured to deliver a guidewire 20 to the occlusion in the blood vessel. In some embodiments, the therapy device 10 includes a guide sheath 22 configured to deliver the treatment catheter 12 and the associated guidewire 20 to the occlusion. In order to secure the guidewire 20 to the treatment catheter 12, a plurality of eyelets 24 (three of which are shown in FIG. 1) are disposed on an exterior of the treatment catheter 12 and configured to secure the guidewire 22 to the treatment catheter 12. For example, each eyelet 24 can comprise a plastic, such as PEBAX.

The eyelets 24 can be positioned or disposed on the treatment catheter 12 in a variety of manners. In some examples, the eyelets 24 are disposed on an exterior of the treatment catheter 12 along a portion of the length of the treatment catheter 12 located between a proximal end 26 of the treatment catheter 12 and the guidewire port 16. In another example, no eyelet 24 is disposed between the guidewire port 16 and the distal end 18 of the treatment catheter 12. In another example, the eyelets 24 are equally spaced along the treatment catheter 12. In another example, the eyelets 24 are spaced from each other along the length of the treatment catheter 12 at an interval of 5-10 cm. These are merely illustrative examples, and should not be construed as limiting.

The eyelets 24 can be secured to the treatment catheter 12 in any suitable manner. In one embodiment, as shown in FIG. 1, each eyelet 24 comprises a loop surrounding the exterior of the treatment catheter 12. A portion of each eyelet 24 can be thermally fused to the exterior of the treatment catheter 12. In another embodiment, as shown in FIG. 2, the eyelets 24 comprise a partial cutout of a portion of the exterior of the treatment catheter 12.

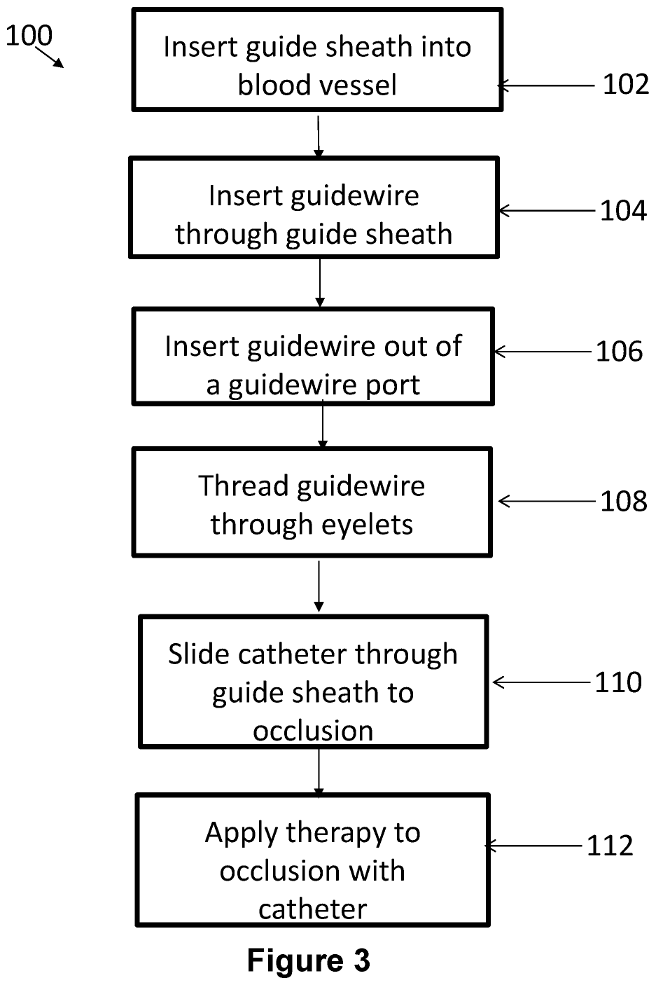

Referring to FIG. 3, an illustrative embodiment of an intravascular therapy method or process 100 using the device 10 is diagrammatically shown as a flowchart. To begin the method 100, a crossing guidewire (not shown) is inserted at least up to the occlusion in the blood vessel. At an operation 102, the guide sheath 22 is inserted in a blood vessel of a patient and to an occlusion in the blood vessel. At an operation 104, the crossing guidewire is removed, and the guidewire 20 is inserted through the inserted guide sheath 22 at least up to the occlusion in the blood vessel and over the crossing guidewire. At an operation 106, a proximal end of the guidewire 20 is inserted into the distal end 18 of the treatment catheter 12, through the guidewire lumen 14, and out the guidewire port 16. At an operation 108, after exiting the guidewire port 16, the proximal end of the guidewire 20 is threaded through the eyelets 24. At an operation 110, after the guidewire 20 is threaded through the eyelets 24, the treatment catheter 12 is slide through the guide sheath 22 along the guidewire 20 toward the occlusion in the blood vessel. At an operation 112, after the sliding, therapy is performed on the occlusion using the treatment catheter 12.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An intravascular therapy device, comprising:
a treatment catheter having a guidewire lumen extending between a guidewire port of the treatment catheter and a distal end of the treatment catheter, a length of the guidewire lumen being less than a length of the treatment catheter; and
a plurality of eyelets disposed on an exterior of the treatment catheter and configured to secure an associated guidewire to the treatment catheter.

2. The intravascular therapy device of claim 1, wherein no eyelet of the plurality of eyelets is disposed between the guidewire port and the distal end of the treatment catheter.

3. The intravascular therapy device of claim 1, wherein a ratio of the length of the guidewire lumen to the length of the treatment catheter is 0.5 or less.

4. The intravascular therapy device of claim 1, wherein the length of the treatment catheter is at least 30 cm, and the length of the guidewire lumen is 15 cm or less.

5. The intravascular therapy device of claim 2, wherein the plurality of eyelets are equally spaced along the treatment catheter.

6. The intravascular therapy device of claim 5, wherein the plurality of eyelets are spaced from each other along the length of the treatment catheter at an interval of 5-10 cm.

7. The intravascular therapy device of claim 1, wherein the eyelets comprises a plastic.

8. The intravascular therapy device of claim 1, wherein each eyelet comprises a loop surrounding the exterior of the treatment catheter.

9. The intravascular therapy device of claim 8, wherein a portion of each eyelet is thermally fused to the exterior of the treatment catheter.

10. The intravascular therapy device of claim 1, wherein the at least one eyelet comprises a partial cutout of a portion of the exterior of the treatment catheter.

11. The intravascular therapy device of claim 1, wherein the treatment catheter comprises a plastic.

12. The intravascular therapy device of claim 1, wherein the treatment catheter and the at least one eyelet comprise PEBAX.

13. The intravascular therapy device of claim 1, further comprising a guide sheath configured to deliver the treatment catheter and the associated guidewire to the occlusion.

14. An intravascular therapy device, comprising:
a treatment catheter having a guidewire lumen extending between a guidewire port of the treatment catheter and a distal end of the treatment catheter, the guidewire lumen providing a path between the guidewire port and the distal end of the treatment catheter, a length of the guidewire lumen being less than one-half of a length of the treatment catheter; and
a plurality of eyelets disposed on an exterior of the treatment catheter along a portion of the length of the treatment catheter located between a proximal end of the treatment catheter and the guidewire port.

15. The intravascular therapy device of claim 14, wherein no eyelet of the plurality of eyelets is disposed between the guidewire port and the distal end of the treatment catheter.

16. The intravascular therapy device of claim 14, wherein a ratio of the length of the guidewire lumen to the length of the treatment catheter is 0.5 or less.

17. The intravascular therapy device of claim 14, wherein the length of the treatment catheter is at least 30 cm, and the length of the guidewire lumen is 15 cm or less.

18. The intravascular therapy device of claim 15, wherein the plurality of eyelets are equally spaced along the treatment catheter.

19. The intravascular therapy device of claim 18, wherein the plurality of eyelets are spaced from each other along the length of the treatment catheter at an interval of 5-10 cm.

20. An intravascular therapy method, comprising:
inserting a guide sheath in a blood vessel of a patient and to an occlusion in the blood vessel;
inserting a guidewire through the inserted guide sheath at least up to the occlusion in the blood vessel;
inserting a proximal end of the guide wire into a distal end of a treatment catheter, through a guidewire lumen of the treatment catheter, and out a guidewire port of the treatment catheter, wherein a length of the guidewire lumen is less than a length of the treatment catheter;
after exiting the guidewire port, threading the proximal end of the guidewire through a plurality of eyelets disposed on an exterior of the treatment catheter;
after the threading, sliding the treatment catheter through the guide sheath along the guidewire toward the occlusion in the blood vessel; and
after the sliding, performing a therapy on the occlusion using the treatment catheter.
